(19) United States
(12) Patent Application Publication
(10) Pub. No.: US 2024/0091247 A1
(43) Pub. Date: Mar. 21, 2024

(54) INHIBITOR FOR CHRONIC MYELOID LEUKEMIA STEM CELLS

(71) Applicants: Ohara Pharmaceutical Co., Ltd., Koka-shi Shiga (JP); Saga University, Saga-shi, Saga (JP)

(72) Inventors: Yuki Kurahashi, Koka-shi Shiga (JP); Shinya Kimura, Saga-shi Saga (JP); Tatsuro Watanabe, Saga-shi Saga (JP); Yuta Yamamoto, Saga-shi Saga (JP); Hiroshi Ureshino, Saga-shi Saga (JP); Kazuharu Kamachi, Saga-shi Saga (JP)

(21) Appl. No.: 18/271,829

(22) PCT Filed: Feb. 21, 2022
(86) PCT No.: PCT/JP2022/006831

§ 371 Date: Jul. 11, 2023

(30) Foreign Application Priority Data
Feb. 23, 2021 (JP) 2021-026848

(51) Int. Cl. A61K 31/706; A61K 31/496; A61K 31/5025; A61K 31/506; A61P 35/02; C12Q 1/6886; G01N 33/68

(52) U.S. Cl. CPC A61K 31/706; A61K 31/496; A61K 31/5025; A61K 31/506; A61P 35/02; C12Q 1/6886; G01N 33/68; C12Q 2600/158; G01N 2333/4704; G01N 2800/52; G01N 2800/54

(57) ABSTRACT

The present invention relates to an inhibitor against the stem cells of chronic myeloid leukemia (CML), a pharmaceutical composition for the treatment of CML having preventive effect on CML recurrence, a method of preventing CML recurrence, and a method of assessing the efficacy of treatment with a drug in a patient with CML, including a step of determining the expression level of latexin.

### TECHNICAL FIELD

The present invention relates to an inhibitor against the stem cells of chronic myeloid leukemia (referred to as CML hereinafter), a pharmaceutical composition for the treatment of CML having preventive effect on CML recurrence, a method of preventing CML recurrence, and a method of assessing the efficacy of treatment with a drug in a patient with CML, including a step of determining the expression level of latexin.

### BACKGROUND

CML is a kind of myeloproliferative tumor in adults. BCR-ABL1, which increases CML cells by activating enzymes called tyrosine kinases, is known as the causative gene of CML onset. The prognosis of patients with CML has been improved due to advent of tyrosine kinase inhibitors (also referred to as TKIs hereinafter) including imatinib (also referred to as IM hereinafter). However, since complete remission of the disease cannot be achieved by treatment with TKIs, the patients need to take the medication over their lifetime, during which they are required to withstand high medical costs and side effects due to long-term administration. Clinical trials where patients confirmed with long-term therapeutic effects of TKIs cease the medication were carried out in recent years (1, 2). However, approximately 40% of the patients confirmed with negative causative gene of CML after taking imatinib for more than 2 years do not relapse even after they ceased the medication, whereas cases of recurrence were also seen (1). In this regard, it has been reported that TKIs cannot be expected to have therapeutic effects on the stem cells of CML (3). Meanwhile, a factor called latexin (also referred to as LXN hereinafter) has been reported to negatively control the maintenance of stem cells in normal hematopoietic stem cells (4). It has also been reported that LXN expression is inhibited in leukemic cells and controlled by DNA methylation (5).

A TKI is a molecular target drug that targets directly the constant tyrosine kinase activity of BCR-ABL1. The medication of a TKI dramatically improves the therapeutic effect in patients with CML. However, therapeutic-resistant recurrence of CML in a patient with CML who has been treated with a TKI, where the TKI does not exhibit its effect any more, has become a clinically significant problem.

The stem cells of CML serve as a source of CML cells. Normal hematopoietic stem cells are known as the origin of the generation of the stem cells of CML. The stem cells of CML remain survival in a dormant state with low proliferative activity and are resistant to TKIs. After treatment, the remaining stem cells of CML may be reactivated. Thus, the stem cells of CML are being recognized as essential targets in cancer therapy. However, the origin, function, and characteristics of the stem cells of CML as well as the molecular mechanisms of therapeutic resistance have not yet been elucidated in details. In addition, neither an inhibitor against the stem cells of CML nor an inhibition method against them, which are clinically applicable, has been reported. Accordingly, development of a therapeutic drug and method that eradicate the stem cells of CML is highly anticipated for radical cure of CML.

### PRIOR ART LITERATURES

### Non-Patent Literatures

1. Mahon F-X, Discontinuation of imatinib in patients with chronic myeloid leukemia who have maintained complete molecular remission for at least 2 years: the prospective, multicentre Stop Imatinib (STIM) trial. Lancet Oncol. 2010; 11(11): 1029-1035.

2. Okada M, et al. Final 3-year Results of the dasatinib discontinuation trial in patients with chronic myeloid leukemia who received dasatinib as a second-line treatment. Clin Lymphoma Myeloma Leuk. 2018; 18(5): 353-360.

3. Corbin A S, et al. Human chronic myeloid leukemia stem cells are insensitive to imatinib despite inhibition of BCR-ABL activity. J Clin Invest. 2011; 121(1): 396-409

4. Ying Liang Y, et al. The quantitative trait gene latexin influences the size of the hematopoietic stem cell population in mice. Nat Genet. 2007; 39(2): 178-188.

5. Liu Y, et al. Latexin is down-regulated in hematopoietic malignancies and restoration of expression inhibits lymphoma growth. PLoS One. 2012; 7(9): e44979.

### SUMMARY OF THE INVENTION

### Problems to be Solved by the Invention

The object of the present invention is to provide therapeutic agents and methods and the like for CML targeting the stem cells of CML.

### Means for Solving the Problems

In order to prevent the recurrence that occurs after treatment is discontinued followed by amelioration due to treatment of CML, the present inventors have made intensive researches to find therapeutic methods targeting the stem cells of CML. As a result, OR21, the first orally available single compound prodrug of desitabine, an inhibitor of DNA methyltransferase, was found not only to inhibit CML stem cells as monotherapy and enhance the antitumor effect of TKI as combination therapy, but also to inhibit stem cells by increasing expression of LTX, a negative regulator of hematopoietic stem cells. The present inventors made further investigations in details based on these findings and completed the present invention.

The present invention solves the above problems by providing the following inventions.

[1] An inhibitor against stem cells of CML comprising a compound represented by formula (I) or a salt thereof:

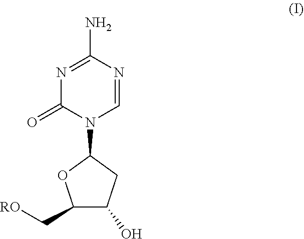

wherein R is a silyl group represented by formula (II):

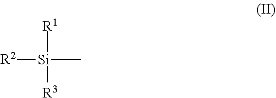

wherein each of R¹, R², and R³ is an alkyl group which may have a substituent.

[2] The inhibitor according to [1], wherein the alkyl group is a methyl, ethyl, or propyl group.

[3] The inhibitor according to [2], wherein the alkyl group is an ethyl group.

[4] The inhibitor according to [1], wherein the compound represented by formula (I) is OR21 (a compound wherein R in formula (I) is a triethylsilyl group).

[5] A pharmaceutical composition for treating CML, comprising a compound represented by formula (I) or a salt thereof:

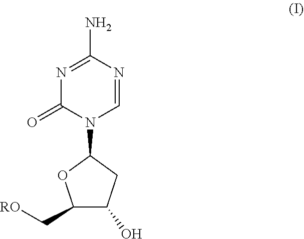

wherein R is a silyl group represented by formula (II):

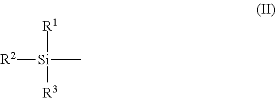

wherein each of R¹, R², and R³ is an alkyl group which may have a substituent, wherein the pharmaceutical composition has an effect of inhibiting the stem cells of CML and prevents the recurrence of CML.

[6] The pharmaceutical composition according to [5], wherein the alkyl group is a methyl, ethyl, or propyl group.

[7] The pharmaceutical composition according to [6], wherein the alkyl group is an ethyl group.

[8] The pharmaceutical composition according to [5], wherein the compound represented by formula (I) is OR21 (a compound wherein R in formula (I) is a triethylsilyl group).

[9] The pharmaceutical composition according to any of [5] to [8], in combination with a TKI.

[10] The pharmaceutical composition according to [9], wherein the TKI is one or more selected from the group consisting of imatinib, gefitinib, erlotinib, sorafenib, dasatinib, sunitinib, lapatinib, nilotinib, pazoponib, crizotinib, ruxolitinib, vandertinib, vemurafenib, axitinib, bosutinib, canonzantinib, ponatinib, regorafenib, tofacitinib, afatinib, dabrafenib, ibrutinib, trametinib, ceritinib, nintedanib, lenvatinib, palbocitinib, carbozantinib, aclabrutinib, brigatinib, neratinib, dacomitinib, gilteritinib, larotrectinib, lorlatinib, and osimertinib.

[11] The pharmaceutical composition according to [9], wherein the TKI is one or more selected from the group consisting of imatinib, nilotinib, dasatinib, bosutinib, and ponatinib.

[12] The pharmaceutical composition according to [9], wherein the compound represented by formula (I) is OR21 (a compound wherein R in formula (I) is a triethylsilyl group), and wherein the TKI is one or more selected from the group consisting of imatinib, nilotinib, dasatinib, bosutinib, and ponatinib.

[13] The pharmaceutical composition according to any of [9] to [12], wherein the compound represented by formula (I) or a salt thereof is administered after administration of the TKI in treating a patient with CML.

[14] The pharmaceutical composition according to any of [9] to [13], wherein the administration comprises oral administration, parenteral administration, or a combination thereof.

[15] The pharmaceutical composition according to any of [9] to [14], wherein the compound represented by formula (I) or a salt thereof is administered orally and the TKI is administered orally or parenterally.

[16] The pharmaceutical composition according to any of [5] to [15] for preventing the recurrence of CML that occurs after treatment with a TKI is discontinued followed by amelioration due to the treatment of CML with the drug.

[17] A therapeutic method for CML, comprising a step of administering to a patient in need of the treatment of CML, a pharmaceutically effective amount of a compound represented by formula (I) or a salt thereof:

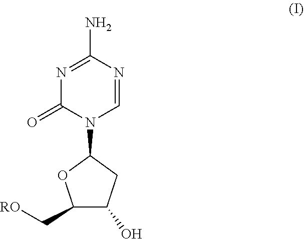

wherein R is a silyl group represented by formula (II):

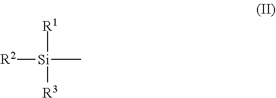

wherein each of R¹, R², and R³ is an alkyl group which may have a substituent, wherein the method prevents the recurrence of CML based on the effect of inhibiting the stem cells of CML.

[18] The therapeutic method according to [17], wherein the alkyl group is a methyl, ethyl, or propyl group.

[19] The therapeutic method according to [18], wherein the alkyl group is an ethyl group.

[20] The therapeutic method according to [17], wherein the compound represented by formula (I) is OR21 (a compound wherein R in formula (I) is a triethylsilyl group).

[21] The therapeutic method according to any of to [20], in combination with a TKI.

[22] The therapeutic method according to [21], wherein the TKI is one or more selected from the group consisting of imatinib, gefitinib, erlotinib, sorafenib, dasatinib, sunitinib, lapatinib, nilotinib, pazoponib, crizotinib, ruxolitinib, vandertinib, vemurafenib, axitinib, bosutinib, canonzantinib, ponatinib, regorafenib, tofacitinib, afatinib, dabrafenib, ibrutinib, trametinib, ceritinib, nintedanib, lenvatinib, palbocitinib, carbozantinib, aclabrutinib, brigatinib, neratinib, dacomitinib, gilteritinib, larotrectinib, lorlatinib, and osimertinib.

[23] The therapeutic method according to [21], wherein the TKI is one or more selected from the group consisting of imatinib, nilotinib, dasatinib, bosutinib, and ponatinib.

[24] The therapeutic method according to [21], wherein the compound represented by formula (I) is OR21 (a compound wherein R in formula (I) is a triethylsilyl group), and wherein the TKI is one or more selected from the group consisting of imatinib, nilotinib, dasatinib, bosutinib, and ponatinib.

[25] The therapeutic method according to any of [21] to [24], wherein the compound represented by formula (I) or a salt thereof is administered after administration of the TKI in treating a patient with CML.

[26] The therapeutic method according to any of [21] to [25], wherein the administration comprises oral administration, parenteral administration, or a combination thereof.

[27] A therapeutic method according to any of [21] to [26], wherein the compound represented by formula (I) or a salt thereof is administered orally and the TKI is administered orally or parenterally.

[28] A therapeutic method according to any of to for preventing the recurrence of CML that occurs after treatment with a TKI is discontinued followed by amelioration due to the treatment of CML.

[29] A method of assessing the efficacy of treatment with a drug in a patient with CML, comprising a step of determining the expression level of LXN in a sample obtained from the patient during or after the treatment with the drug, a step of comparing these levels of expression, and a step of assessing the efficacy of the treatment with the drug, wherein
(1) when the expression level of LXN during or after the treatment with the drug has increased compared to that before the treatment, it is assessed that the treatment with the drug in the patient is effective and the treatment with the drug can be discontinued or terminated without recurrence of the disease,
(2) when the expression level of LXN during or after the treatment with the drug does not increase compared to that before the treatment, it is assessed that the disease will relapse after the treatment with the drug is discontinued or terminated, or
(3) when the assessment of (2) is made, it is assessed that it is effective in preventing recurrence of the disease to continue the treatment with the drug in combination with a drug having an inhibitory effect on the stem cells of CML, or continue the treatment with a drug having an inhibitory effect on the stem cells of CML.

[30] The method according to [29], wherein the sample obtained from a patient is bone marrow or peripheral blood.

[31] The method according to any of [29] to [30], wherein the increase in the expression level of LXN during or after treatment is 1.5 times or more, preferably 2.0 times or more compared to that before the treatment.

[32] The method according to any of [29] to [31], wherein the expression level of LXN is the mRNA expression level of LXN.

[33] The method according to any of [29] to [32], wherein the mRNA expression level is determined using a method selected from the group consisting of RT-PCR, gene expression profiling, and microarray analysis.

[34] The method according to any of [29] to [33], wherein the expression level of LXN is the protein expression level of LXN.

[35] The method according to any of [29] to [34], wherein the protein expression level is determined using a method selected from the group consisting of immunohistochemistry, immunofluorescence, mass spectrometry, flow cytometry, and Western blotting.

[36] The method according to any of [29] to [35], wherein the drug used in the treatment of CML is a TKI or OR21 (a compound wherein R in formula (I) is a triethylsilyl group).

[37] The method according to any of [29] to [36], wherein the drug having an inhibitory effect on the stem cells of CML is OR21 (a compound wherein R in formula (I) is a triethylsilyl group).

### Effects of the Invention

According to the present invention, an inhibitor against the stem cells of CML comprising a compound represented by formula (I) or a salt thereof, a pharmaceutical composition for the treatment of CML which prevents its recurrence, and a method of preventing recurrence of CML can be provided.

According to the present invention, it has been found that OR21 exhibits an anti-tumor effect as a monotherapy and enhances the anti-tumor effect of a TKI and inhibits the stem cells of CML as a combination therapy. Combination therapy of TKI and OR21 is expected as a promising therapeutic method (treatment-free remission: TFR) for CML.

### BRIEF DESCRIPTION OF DRAWINGS

FIG. 1 shows the effects of a DNA methyltransferase inhibitor and a tyrosine kinase inhibitor on the stem cells or progenitor cells of CML in mouse models of CML.

FIG. 2 shows the effect of a DNA methyltransferase inhibitor on the stem cells or progenitor cells of CML in secondarily transplanted mouse models of CML.

FIG. 3 shows the gene expression level of LXN in patients with CML and healthy subjects.

FIG. 4 shows the effects of a DNA methyltransferase inhibitor and a tyrosine kinase inhibitor on gene expression and protein expression of LXN in K562 cells and KBM5 cells obtained by comprehensively analyzing gene expression with microarrays.

FIG. 5 shows the effects of a DNA methyltransferase inhibitor and a tyrosine kinase inhibitor on gene expression and protein expression of LXN in K562 cells and KBM5 cells.

FIG. 6 shows the effects of a DNA methyltransferase inhibitor and a tyrosine kinase inhibitor on gene expression and protein expression of LXN in K562 cells and KBM5 cells.

FIG. 7 shows the gene expression level of LXN in patients with CML and healthy subjects.

FIG. 8 shows the effects of a DNA methyltransferase inhibitor and a tyrosine kinase inhibitor on the stem cells of CML.

### DETAILED DESCRIPTION OF THE INVENTION

Unless stated otherwise, the terms used in the specification and claims have the meanings set forth below.

Generally, the nomenclature used herein, as well as procedures of experiments in organic chemistry, drug chemistry, and pharmacology described herein are well known and commonly used in the art. Unless otherwise defined, all technical and scientific terms used herein have the same meaning as commonly understood by those skilled in the art to which this disclosure belongs.

The term “stem cells of CML” refers to the cells that are present in a blood tumor and have replication competence, multipotency, and hematological tumorigenicity.

The term “an inhibitor against the stem cells of CML”, also referred to as an inhibitor or remover against the stem cells of CML, refers to a drug that targets the stem cells of CML and exhibits inhibitory effect on the proliferation of the stem cells of CML or cytotoxic effect on the stem cells of CML. In addition, an inhibitor against the stem cells of CML may also be a drug that inhibits at least one or more than two functions of the stem cells of CML, such as replication competence, multipotency, and hematological tumorigenicity.

The term “a subject” refers to animals including, but not limited to, primates (e.g., humans), cows, pigs, sheep, goats, horses, dogs, cats, rabbits, rats, and mice. The terms “a subject” and “a patient” are used herein interchangeably with respect to a mammalian subject, such as, a human. In one embodiment, they are used interchangeably with respect to a human.

The terms “treat”, “treating”, and “treatment” mean reducing or inhibiting a disorder, a disease or condition or one or more symptoms associated there with, or reducing or eradicating the cause itself of a disorder, a disease or condition.

The terms “prevent”, “preventing”, and “prevention” mean delaying and/or eliminating the onset of a disorder, a disease or condition and/or symptoms associated there with, preventing the acquisition of a disorder, a disease or condition, or reducing the risk of acquiring a disorder, a disease or condition.

When a compound is administered, the term “therapeutically effective amount” means the amount of the compound which is sufficient to prevent or reduce to some extent the onset of one or more symptoms of a disorder, a disease, or condition to be treated. In addition, the term “therapeutically effective amount” refers to the amount of a compound which is sufficient to induce a biological or medical reaction of biological molecules (e.g., protein, enzyme, RNA or DNA), cells, tissues, systems, animals, or human beings, for which researchers, veterinarians, physicians, or clinicians are searching.

The term “relapsed” refers to a condition in a subject or mammal who had been in amelioration from cancer after treatment where the recovery of cancer cells was compromised.

### An Inhibitor Against the Stem Cells of CML

The present invention provides an inhibitor against the stem cells of CML comprising a compound represented by formula (I) or a salt thereof:

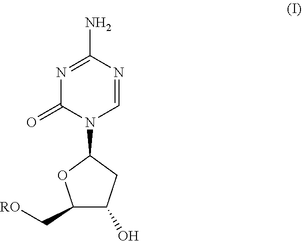

wherein R is a silyl group represented by formula (II):

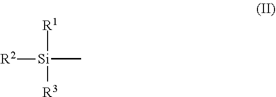

wherein each of R¹, R², and R³ is an alkyl group which may have a substituent.

“An alkyl group” refers to, unless otherwise limited, a saturated aliphatic hydrocarbon group such as a linear, branched, or cyclic alkyl group having 1 to 8 carbon atoms. Examples thereof include C₁-C₆ alkyl groups such as methyl, ethyl, propyl, isopropyl, butyl, sec-butyl, isobutyl, tert-butyl, pentyl, and hexyl groups, heptyl, 2-methylhexyl, 5-methylhexyl, 2,2-dimethylpentyl, 4,4-dimethylpentyl, 2-ethylpentyl, 1,1,3-trimethylbutyl, 1,2,2-trimethylbutyl, 1,3,3-trimethylbutyl, 2,2,3-trimethylbutyl, 2,3,3-trimethylbutyl, 1-propylbutyl, 1,1,2,2-tetramethylpropyl, octyl, 2-methylheptyl, 3-methylheptyl, 6-methylheptyl, 2-ethylhexyl, 5,5-dimethylhexyl, 2,4,4-trimethylpentyl, 1-ethyl-1 -methylpentyl, cyclopropyl, cyclobutyl, cyclopentyl, cyclohexyl, cycloheptyl, cyclooctyl groups, and the like, preferably, C₁-C₆ alkyl groups. Preferable examples of C₁-C₆ alkyl groups are methyl, ethyl, and propyl groups. More preferable example of C₁-C₆ alkyl groups is ethyl group. Preferable examples of cyclic alkyl groups are cyclopentyl and cyclohexyl groups.

“An alkyl group which may have a substituent” refer to those which may have a substituent or may not be substituted. In the case of being substituted, there may be 1 to 5 substituents, preferably 1 to 3 substituents at possible positions of the alkyl group. When the number of substituents is 2 or more, each substituent may be the same or different. Examples of the substituents include halogen atoms, cyano and nitro groups, etc., preferably halogen atoms.

“A halogen atom” refers to fluorine, chlorine, bromine, or iodine atoms, and the like, preferable examples thereof are fluorine and chlorine atoms.

Among the compounds represented by formula (I), a compound in which R is a triethylsilyl group (referred to as OR21 hereinafter) is preferable. OR21 is known and has the following structure:

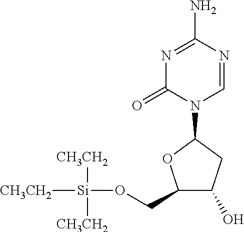

The compound represented by formula (I) and OR21 can be prepared, isolated, or obtained by any method known to those skilled in the art. For example, they can be prepared according to the methods described in Japanese Patent No. 6162349, the disclosure of which is incorporated herein by reference in its entirety.

A salt of the compound represented by formula (I) of the present invention may be any salt as long as it is pharmacologically acceptable. The examples of which include, but are not limited to, acid addition salts from inorganic acids (e.g., hydrochloride, sulfate, hydrobromide, and phosphate, etc.) and those from organic acids (e.g., acetate, trifluoroacetate, succinate, maleate, fumarate, propionate, citrate, tartrate, lactate, oxalate, methanesulfonate, and p-toluenesulfonate, etc.).

The compound represented by formula (I) or a salt thereof may be a crystal, which may be a single crystal form or a mixture of a plurality of crystalline forms. The crystals can be produced by crystallization by applying a crystallization process known per se.

In addition, the compound represented by formula (I) or a salt thereof may be a solvate (e.g., a hydrate or the like), and any of the solvate and the non-solvate (e.g., a non-hydrate, etc.) is included in the compound represented by formula (I) or a salt thereof.

In the present invention, since OR21 exhibits an effect of inhibiting the progenitor cells of CML in a mouse model of CML and an effect of inhibiting the stem cells of CML that increases by administration of IM, it is shown to be extremely useful and effective as a novel therapeutic agent for CML.

### A Pharmaceutical Composition

The present invention provides a pharmaceutical composition for treating CML, comprising a compound represented by formula (I) or a salt thereof, the pharmaceutical composition having an effect of inhibiting the stem cells of CML and preventing the recurrence of CML.

In addition, the present invention provides a pharmaceutical composition for treating CML, comprising a compound represented by formula (I) or a salt thereof in combining with a TKI, the pharmaceutical composition having an effect of inhibiting the stem cells of CML and preventing the recurrence of CML.

The TKI used in the present invention includes, for example, imatinib, gefitinib, erlotinib, sorafenib, dasatinib, sunitinib, lapatinib, nilotinib, pazoponib, crizotinib, ruxolitinib, vandertinib, vemurafenib, axitinib, bosutinib, canonzantinib, ponatinib, regorafenib, tofacitinib, afatinib, dabrafenib, ibrutinib, trametinib, ceritinib, nintedanib, lenvatinib, palbocitinib, carbozantinib, aclabrutinib, brigatinib, neratinib, dacomitinib, gilteritinib, larotrectinib, lorlatinib, osimertinib, and the like. Preferably, the TKI is one or more selected from the group consisting of imatinib, nilotinib, dasatinib, bosutinib, and ponatinib.

When the pharmaceutical composition of the present invention is administered to a patient as a pharmaceutical formulation, the compound represented by formula (I) may be formulated alone or mixed with a TKI and a pharmaceutically acceptable carrier etc. The content of the compound represented by formula (I) in a pharmaceutical formulation is usually 0.1-100% (w/w). In addition, when a concomitant drug is used as a pharmaceutical formulation, the content of the compound represented by formula (I) is usually 0.1-99.9% (w/w).

A suitable pharmaceutical composition for use in the present invention contains active ingredients which present in an effective amount for achieving a therapeutic and/or prophylactic purpose for a symptoms being treated.

The pharmaceutical compositions used in the present invention are provided as dosage forms for oral administration. A pharmaceutical composition provided herein may be provided in solid, semi-solid, or liquid dosage forms for oral administration. As used herein, the oral administration includes oral and sublingual administrations. Suitable dosage forms for oral administration include, but are not limited to, tablets, capsules, pills, troches, medicinal candy, fragrance formulations, cachets, pellets, drug-added chewing gum, granules, bulk, foamed formulations or non-foamed powders or granules, solutions, emulsions, suspensions, wafers, sprinkles, elixirs, and syrups. In addition to the active ingredients, a pharmaceutical composition further comprises one or more pharmaceutically acceptable additives. Additives include, but are not limited to, carriers, excipients, binders, fillers, diluents, disintegrants, wetting agents, lubricants, flow promoters, colorants, dye migration inhibitors, sweetening, flavoring agents, and the like.

The amount of the compound represented by formula (I) in a pharmaceutical composition or dosage form may be in the range of, for example, about 1-2,000 mg, about 10-2,000 mg, about 20-2,000 mg, about 50-1,000 mg, about 100-500 mg, about 150-500 mg, or about 150-250 mg.

When a compound of the present invention is used as a therapeutic agent for the stem cells of CML, its effective dosage may be selected as appropriate depending on nature of CML, progression degree of CML, therapeutic strategy, extent of metastasis, tumor volume, body weight, age, sex, and (genetic) ethnic background of a patient, etc. However, a pharmaceutically effective amount is generally determined based on factors such as clinically observed symptoms and progression degree of CML, etc. The daily dosage is, for example, about 0.01-10 mg/kg (about 0.5-500 mg in case of an adult having a body weight of 60 kg), preferably about 0.05-5 mg/kg, more preferably about 0.1-2 mg/kg. The administration may be carried out one time or multiple times.

The pharmaceutical composition can be produced according to a conventional method in the formulation art, such as one described in Japanese Pharmacopoeia.

### Therapeutic Methods

The present invention provides a therapeutic method for CML, comprising a step of administering to a patient in need of the treatment of CML, a pharmaceutically effective amount of a compound represented by formula (I) or a salt thereof, the method preventing the recurrence of CML based on the effect of inhibiting the stem cells of CML.

In addition, the present invention provides a therapeutic method for CML, comprising a step of administering to a patient in need of the treatment of CML, a pharmaceutically effective amount of a compound represented by formula (I) or a salt thereof in combining with a TKI, the method preventing the recurrence of CML based on the effect of inhibiting the stem cells of CML.

When a compound represented by formula (I) of the present invention is combined with a TKI, the timing of administration of the compound represented by formula (I) and the TKI is not limited. Both may be administered to a subject simultaneously or at a time interval. A compound represented by formula (I) and a TKI may be formulated separately or may be a drug combination wherein both are mixed. The dosage of a concomitant drug may be based on a clinically used dosage and may be selected appropriately depending on the subject to be administered, route of administration, disease, and combination thereof, etc. The dosage of a concomitant drug may be, for example, one third to three times of that when the concomitant drug is used as a single drug.

The dosage form of a compound represented by formula (I) of the present invention and a TKI is not particularly limited. They may be combined when administered. Examples of such dosage form include (1) administration of a single formulation obtained by simultaneously formulating a compound represented by formula (I) and a TKI, (2) simultaneous administration of two formulations obtained by separately formulating a compound represented by formula (I) and a TKI via the same administration route, (3) administration at a time interval of two formulations obtained by separately formulating a compound represented by formula (I) and a TKI via the same administration route, (4) simultaneous administration of two formulations obtained by separately formulating a compound represented by formula (I) and a TKI via different administration routes, and (5) administration at a time interval of two formulations obtained by separately formulating a compound represented by formula (I) and a TKI via different administration routes (e.g., an administration order: firstly a TKI and secondly a compound of formula (I), or vice versa).

Following excellent effects can be achieved by combining a compound represented by formula (I) of the present invention with a TKI:
(1) The dosage can be reduced as compared to a separate administration of a compound represented by formula (I) or a TKI.
(2) The type of concomitant drug can be selected depending on the symptoms (mild or severe, etc.) of a patient.
(3) A long term treatment can be set by selecting a TKI having a different mechanism of action with a compound represented by formula (I).
(4) Persistent therapeutic effect can be achieved by selecting a TKI having a different mechanism of action with a compound represented by formula (I).
(5) Synergistic therapeutic effect can be achieved by combining a compound represented by formula (I) with a TKI.
(6) Preventive effect of the recurrence after discontinuation of administration due to amelioration followed by treatment of CML can be achieved by combining a compound represented by formula (I) with a TKI.

In one embodiment, the present invention provides the inhibitor against the stem cells of CML for preventing the recurrence of CML.

In one embodiment, the present invention provides the inhibitor against the stem cells of CML for producing a drug which prevents the recurrence of CML.

### A Method of Assessing the Efficacy of a Drug in Treating a Patient with CML

The present invention provides a method of assessing the efficacy of treatment with a drug in a patient with CML, comprising a step of determining the expression level of LXN in a sample obtained from the patient before and during or after the treatment with the drug, a step of comparing these levels of expression, and a step of assessing the efficacy of the treatment with the drug, wherein
(1) when the expression level of LXN during or after the treatment with the drug has increased compared to that before the treatment, it is assessed that the treatment with the drug in the patient is effective and the treatment with the drug can be discontinued or terminated without recurrence of the disease,
(2) when the expression level of LXN during or after the treatment with the drug does not increase compared to that before the treatment, it is assessed that the disease will relapse after the treatment with the drug is discontinued or terminated, or
(3) when the prediction of (2) is made, it is assessed that it is effective in preventing recurrence of the disease to continue the treatment with the drug in combination with a drug having an inhibitory effect on the stem cells of CML, or continue the treatment with a drug having an inhibitory effect on the stem cells of CML.

A drug used in a patient with CML in the present invention can be any compound as long as it exhibits an inhibitory effect on the proliferation of the stem cells of CML, a cytotoxic effect on the stem cells of CML, or an effect of enhancing sensitivity of cells to a drug. As effective drugs for the treatment of CML, for example, those included in chemotherapeutic drugs, biological response modifiers, chemical sensitizer, and the like can be exemplified.

A chemotherapeutic drug means a drug that is used to kill cancer cells or delay their proliferation. Thus, both cytotoxic and cytostatic agents are considered as chemotherapeutic drugs.

A biological response modifier means a drug that stimulates or restores the ability of an immune system to combat a disease. Some, but not all, biological response modifiers can delay the proliferation of cancer cells. Thus, they are also considered as chemotherapeutic drugs.

A chemical sensitizer means a drug that increases the sensitivity of tumor cells to the effect of a chemotherapeutic drug.

Examples of drugs for patients with CML in the present invention include, but are not limited to, DNA methyltransferase inhibitors, histone methyltransferase inhibitors, TKIs, p53 gene inhibitors, enzyme inhibitors, and the like.

### DNA Methyltransferase Inhibitors

The DNA methyltransferase is a group of enzymes which methylate the N6-position of adenine, the N4-position or the 5-position of cytosine in DNA chains. Particularly, in the part of sequence called CpG islands and often recognized in a promoter region of an expression gene, the group of enzymes which methylate 5-position of cytosine plays an extremely important role in regulating normal generation and differentiation of cells. Since the DNA methyltransferase has epigenetic effect on gene expression, the inhibitors against the enzymes are used as anticancer drugs.

Examples of the DNA methyltransferase inhibitors used in the present invention include, but are not limited to, a compound represented by formula (I) or a pharmaceutically acceptable salt thereof, decitabine, azacytidine, RG-108, thioguanine, zebularine, SGI-110, SGI-1027, lomeguatrib, and procainamide hydrochloride.

Decitabine has the chemical name of 4-amino-1-(2-deoxy-β-D-erythro-pentofuranosyl)-1,3,5-triazin-2(1H)-one and CAS number of 2353-33-5. Examples of the pharmaceutic adjuvant of decitabine and its metabolic enzyme inhibitor include ASTX727. ASTX727 is a pharmaceutic adjuvant of decitabine and E7727 (generic name: cedazuridine), which is a cytidine deaminase inhibitor. E7727 has the chemical name of (4R)-1-(2-deoxy-2,2-difluoro-beta-D-erythro-pentofuranosyl)-4-hydroxytetrahydropyrimidin-2(1H)-one and CAS number of 1141397-80-9. Azacitidine has the chemical name of 4-amino-1-β-D-ribofuranosyl-s-triazin-2(1H)-one and CAS number of 320-67-2. RG-108 has the chemical name of 2(S)-(1,3-dioxo-2,3-dihydro-1H-isoindol-2-yl)-3-(1H-indol-3-yl)propionic acid and CAS number of 48208-26-0. Tioguanine has the chemical name of 2-amino-1,9-dihydro-6H-purine-6-thione and CAS number of 154-42-7. Zebularine has the chemical name of 1-(β-D-ribofuranosyl)pyrimidin-2(1H)-one and CAS number of 3690-10-6. SGI-110 (generic name: guadecitabine) has the chemical name of 2′-deoxy-5′-O-[(2′-deoxy-5-azacytidin-3′-O-yl)(hydroxy)phosphoryl]guanosine and CAS number of 929904-85-8 (sodium salt). SGI-1027 has the chemical name of N-[4-(2-amino-6-methylpyrimidin-4-ylamino)phenyl]-4-(quinolin-4-ylamino)benzamide and CAS number of 1020149-73-8. Lomeguatrib has the chemical name of 6-[(4-bromo-2-thienyl)methoxy]-7H-purin-2-amine and CAS number of 192441-08-0. These compounds may be in the form of pharmaceutically acceptable salts thereof. The pharmaceutically acceptable salts are exemplified by the above-mentioned salts. The salts may be the anhydrides or solvates described above.

### Histone Methyltransferase Inhibitors

Histone methyltransferase is an enzyme which transfers a methyl group from S-adenosylmethionine of a coenzyme to the amino group of lysine residue of histone 3 (H3) protein. The methylation modification of lysine residue has an epigenetic effect on gene expression, and is thus extremely important for gene expression regulation. Therefore, the inhibitors against histone methyltransferase are used as anticancer drugs.

Examples of histone methyltransferase inhibitors used in the present invention include, but are not limited to, EPZ-6438, DS-3201b, GSK-126, chaetocin, and BIX-01294, etc., preferably EPZ-6438 and DS-3201b.

EPZ-6438 (generic name: tazemetostat) is an inhibitor against histone methyltransferase EZH2. It has the chemical name of N-[(4,6-dimethyl-2-oxo-1,2-dihydropyridin-3-yl) methyl]-5-[ethyl(tetrahydro-2H-pyran-4-yl) amino]-4-methyl-4′-(morpholin-4-ylmethyl) biphenyl-3-carboxamide and CAS number of 1467052-75-0 (hydrobromide salt). DS-3201b (generic name: valemetostat) is a dual inhibitor against histone methyltransferases EZH1 and EZH2. It has the chemical name of 4-methylbenzene-1-sulfonic acid (2R)-7-chloro-2-[(trans)-4-(dimethylamino) cyclohexyl]-N-[(4,6-dimethyl-2-oxo-1,2-dihydropyridin-3-yl) methyl]-2,4-dimethyl-2H-1,3-benzodioxole-5-carboxamide and CAS number of 1809336-39-7 (tosilate). GSK-126 is an inhibitor against histone methyl transferase EZH2. It has the chemical name of 1-[2(S)-butyl]-N-(4,6-dimethyl-2-oxo-1,2-dihydropyridin-3-ylmethyl)-3-methyl-6-[6-(1-piperazinyl)pyridin-3-yl]-1H-indole-4- carboxamide and CAS number of 1346574-57-9. Chaetocin has the chemical name of (3S,3′S,5aR,5′aR,10bR, 10′bR,11aS,11′aS)-1,1′,2,2′,3,3′,4,4′,5a,5′a,6,6′,10b,10b′,11,11′,11a,11a′- octadecahydro-3,3′-bis(hydroxymethyl)-2,2′-dimethyl-[bi-3,11a-epidithio-11aH-pyrazino[1′,2′:1,5]pyrrolo[2,3-b]indole]-1,1′,4,4′-tetraone and CAS number of 28097-03-2. BIX-01294 has the chemical name of N-(1-benzylpiperidin-4-yl)-6,7-dimethoxy-2-(4-methylperhydro-1,4-diazepin-1-yl) quinazolin-4-amine and CAS number of 935693-62-2.

Examples of TKIs include, but are not limited to, imatinib, gefitinib, erlotinib, sorafenib, dasatinib, sunitinib, lapatinib, nilotinib, pazoponib, crizotinib, ruxolitinib, vandertinib, vemurafenib, axitinib, bosutinib, canonzantinib, ponatinib, regorafenib, tofacitinib, afatinib, dabrafenib, ibrutinib, trametinib, ceritinib, nintedanib, lenvatinib, palbocitinib, carbozantinib, aclabrutinib, brigatinib, neratinib, dacomitinib, gilteritinib, larotrectinib, lorlatinib, osimertinib, and the like.

Examples of p53 gene inhibitors or enzyme inhibitors include, but are not limited to, pifithrin, nutlin, DS3201, HBI-8000, trichostatin A (TSA), suramin, EPZ005687, adox, and the like.

In the method of the present invention, “a patient with CML” refers to a patient who has been diagnosed with CML.

The method of the present invention comprises a step of determining the expression level of LXN in a sample obtained from a patient with CML. “A sample” refers to the tissue containing the cells of a patient with CML. Examples of the source of a sample include all tissues in the body, such as blood (whole blood), umbilical cord blood, lymph, tissue fluid (interstitial fluid, intercellular fluid, and interstitial fluid), body cavity fluid (ascites, pleural fluid, pericardial fluid, cerebrospinal fluid, joint fluid, and aqueous humor), nasal drops, etc., preferably bone marrow or peripheral blood, more preferably mononuclear cells of peripheral blood, in view if their low invasiveness. Mononuclear cells of peripheral blood can be obtained from collected whole blood, for example, by the method of Ficoll density gradient centrifugation. Besides, cells expressing or not expressing specific cell surface marker proteins may be separated and collected by positive or negative selection using magnetic beads for cell separation. The cells of a patient with CML may be cell lines established from the cells of the patient.

The “expression level of latexin (or LXN)” is the gene (mRNA) or protein expression level of LXN in a sample. In order to determine the mRNA expression level in a sample, total RNA is usually extracted from a tissue. The methods of which are well known to those skilled in the art. As a method of detecting the mRNA expression level of LXN, any method may be used as long as a part or all of mRNA or single stranded complementary DNA (cDNA) can be detected specifically. Following examples of the methods can be given: a method of extracting the total RNA of cells in a sample and detecting by Northern blotting using a probe consisting of base sequences complementary to mRNA of LXN, a method of synthesizing cDNA from the extracted total RNA using reverse transcriptase and then detecting by a quantitative PCR method such as a competitive PCR (polymerase chain reaction) method or a real-time PCR method, etc., and a method of synthesizing cDNA from the total RNA using reverse transcriptase, then labeling the cDNA with biotin or digoxigenin, etc., indirectly labeling the cDNA with an antibody that recognizes avidin or digoxigenin, which has high affinity for biotin labeled with a fluorescent substance, and then detecting with a microarray using a probe consisting of base sequences complementary to cDNA which are immobilized on a support such as glass, silicon, plastic, etc. that can be used for hybridization, and synthesized from the mRNA of LXN and the mRNA of any reference gene. By gene expression profiling, the mRNA expression of LXN can be profiled, and its relation with signs or symptoms in a patient with CML can also be examined in more details.

In order to determine the protein expression level of LXN in a sample, methods understood by those skilled in the art, such as immunohistochemistry, immunofluorescence, mass spectrometry, flow cytometry, and Western blotting, etc. can be used. As anti-LXN antibodies required for the detection of protein of LXN, those which are commercially available can be used. In mass spectrometry, for a sample solution, it is preferable to use an ionization method such as MALDI-MS (matrix assisted laser desorption ionization mass spectrometry) by which compounds of high molecular weight are less likely to decompose.

In the assessment method of the present invention, when the expression level of LXN during or after treatment with a drug has increased compared to that before the treatment, it can be assessed that the treatment with the drug in the patient is effective and the treatment with the drug can be discontinued or terminated without recurrence of the disease.

In the assessment method of the present invention, when the expression level of LXN during or after treatment with a drug does not increase compared to that before the treatment, it can be assessed that the disease will relapse after the treatment with the drug is discontinued or terminated. When such assessment is made, it is effective in preventing recurrence of the disease to continue the treatment with the drug in combination with a drug having an inhibitory effect on the stem cells of CML, or continue the treatment with a drug having an inhibitory effect on the stem cells of CML.

In the assessment method of the present invention, the increase in the expression level of LXN during or after treatment is 1.5 times or more, preferably 2.0 times or more compared to that before the treatment.

### EXAMPLES

The present invention will be described with examples as follows, although it is not limited to the following examples.

### Example 1

### Effects of DNA Methyltransferase Inhibitors and Tyrosine Kinase Inhibitors on the Stem Cells or Progenitor Cells of CML in Mouse Models of CML

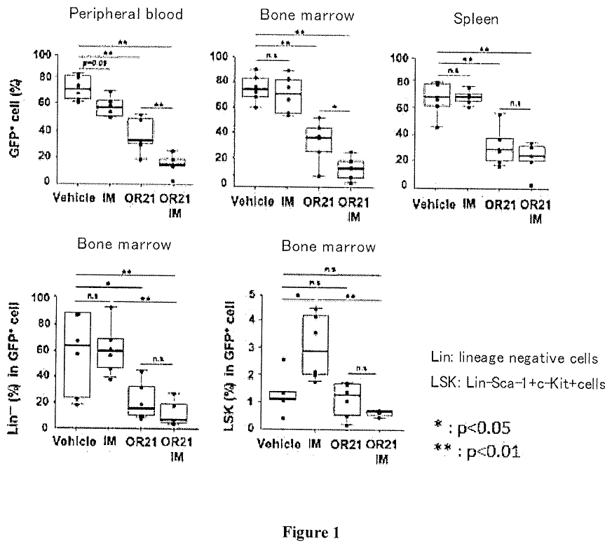

Recipient mice were engrafted with myeloid cells to which GFP positive MIG-BCR-ABL1 had been introduced and used as CML models. They were divided into vehicle administration group (1% DMSO, intraperitoneal administration, n=6), OR21 administration group (1.35 mg/kg, intraperitoneal administration, n=6), imatinib (IM) administration group (150 mg/kg, oral administration, n=6), and OR21 and imatinib (OR21+IM) administration group (n=5). After administration of the drugs to each group for 12 days, the GFP positive cells in peripheral blood, bone marrow, and spleen were determined. The results are shown in FIG. 1. Inter-group comparisons were performed by a significance test of Mann-Whitney U-tests (p<0.05, **p<0.01) in FIG. 1. According to FIG. 1, no decrease in GFP positive cell rate was observed in the IM administration group, whereas a significant decrease was observed in the OR21 administration group or the OR21+IM administration group, compared to the vehicle administration group. In addition, in the OR21 administration group or OR21+IM administration group, the linage-negative cells (Lin-) in bone marrow decreased and Lin-Sca-1+c-Kit+(LSK) cells that increased in the IM administration group decreased. This indicated the inhibitory effects of OR21 against the progenitor cells or stem cells of CML that increase with administration of IM.

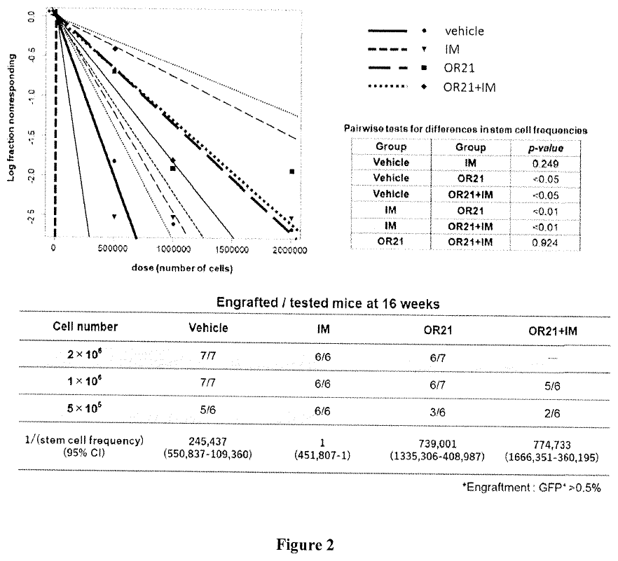

In addition, in order to investigate the effect of OR21 on the stem cells of CML, a limiting dilution assay was carried out using secondarily transplanted mice. Secondary transplantation means transplanting GFP positive cells from each donor mouse of the vehicle group and the four groups administered with the drugs (the OR21 group, IM group, OR21 group, and OR21+IM group) into recipient mice at 2×10⁶, 1×10⁶, or 5×10⁵ cells/mouse (Due to insufficient cell number, the transplantation at 2×10⁶ cell was omitted in the OR21+IM administration group). Then, engraftment of GFP positive cells in the peripheral blood (PB) at 16 weeks after the secondary transplantation was determined and analyzed by limiting dilution assay. The results are shown in FIG. 2. In the table at the bottom of FIG. 2, the number of mice engrafted at each cell number (2×10⁶, 1×10⁶, or 5×10⁵ cells) in each group is shown as the “number of engrafted mice/total number of transplanted mice”. The bottom row of the table indicates the cell number required to be engrafted. The larger numbers mean that it is more difficult to engraft. It is noted that engraftment of GFP positive cells is defined to have a GFP positive rate of 0.5% or more in the peripheral blood of recipient mice. The upper left figure in FIG. 2 represents the results of the table, wherein the horizontal axis represents the number of transplanted cells, the vertical axis represents the non-transplant rate, and the shape of the linear and plots represent each group (vehicle group: a solid line and circles, IM group: a median dotted line and triangles, OR21 group: a rough dotted line and squares, OR21+IM group: a fine dotted line and diamonds). The higher plots or slope of the lines mean that it is more difficult to engraft. The upper right table in FIG. 2 shows the p-values when the inter-group comparison was performed by a significance test of pairwise test. In the mice of the secondary transplantation from the OR21 administration group or OR21+IM administration group, GFP positive cells was significantly reduced compared to the mice of the secondary transplantation from the vehicle administration group or from the IM administration group. Consequently, it was suggested that the administration of OR21 effectively inhibits the progenitor cells or stem cells of CML.

### Example 2

### Gene Expression Levels of LXN in Patients with CML and Healthy Subjects

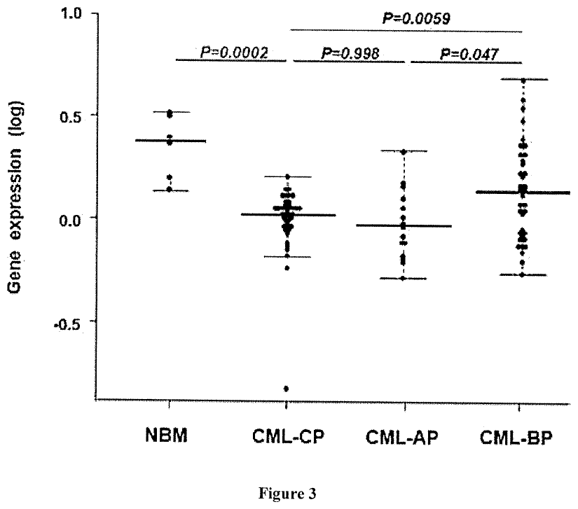

Total RNA was extracted from bone marrow CD34+ cells of patients with CML in chronic phase (CML-CP: 42 cases), patients with CML in aggressive phase (CML-AP: 15 cases), patients with CML in blast phase (CML-BP: 36 cases), and healthy subjects (NBM: 6 cases), respectively and a comprehensive analysis of gene expression level in these cells was carried out using with Merck Human 25 k v2.2.1 microarray. The results have been reported by Radich J P, Dai H, Mao M, Oehler V et al., entitled “Gene expression changes associated with progression and response in chronic myeloid 1 eukemia.” in Proc Natl Acad Sci USA 2006 Feb 21;103(8):2794-9. The present inventors analyzed the results reported by Radich et al. and calculated the mRNA expression level of LXN in the above cells. The results are shown in FIG. 3, wherein, in the graph, the centerline indicates the values of median, the upper and lower bars indicate standard deviations, and the vertical axis indicates the gene expression level converted to common logarithm. It has become clear from FIG. 3 that the mRNA expression level of LXN in patients with CML in any phase shows lower values compared with that in healthy subjects. Particularly, patients with CML in chronic phase have significantly lower values compared to healthy subjects, suggesting that the expression of LXN has been lower than healthy subjects since diagnosis of CML.

### Example 3

### Effects of DNA Methyltransferase Inhibitors and Tyrosine Kinase Inhibitors on Gene Expression and Protein Expression of LXN in K562 cells and KBMS Cells

K562 cells (cell lines derived from CML) were purchased from JCRB cell bank and KBM5 cells (cell lines derived from CML) were provided by Dr. M. Beran (MD Anderson Cancer Center, University of Texas). These calls were cultured in an RPMI 1640 medium containing 10% fetal bovine serum (FBS) and 1% penicillin-streptomycin at 37° C. in atmosphere of 5% CO₂.

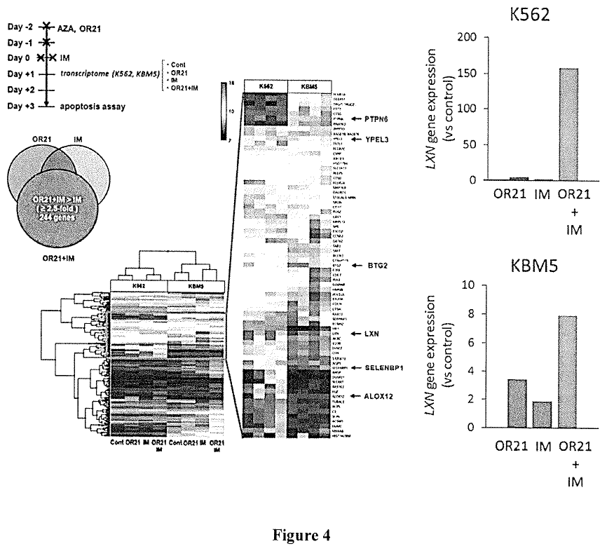

0, 24, and 48 hours after the K562 cells or KBM5 cells were seeded, 100 nM OR21, a DNA methyltransferase inhibitor, was added, and then 1000 nM imatinib, a tyrosine kinase inhibitor, was added and further cultured for another day. In parallel, the cells added only with 100 nM OR21 without imatinib or the cells added only with 1000 nM imatinib without OR21 were also cultured. After the culture, mRNA was collected and gene expression was comprehensively analyzed by microarrays. The results are shown in FIG. 4. As a result of microarray analysis, there were 1,785 genes whose expression was increased by 2.5 times or more compared to control due to the combination of OR21 and imatinib (IM) (OR21+IM). Among these genes, there were 244 genes whose expression was increased by 2.5 times or more compared to the single drug of IM and which were common to the genes of both K562 cells and KBM5 cells. These 244 genes were clustered and analyzed and 71 genes containing cancer suppressor genes (PTPN6, YPEL3, BTG2, LXN, SELENBPI, and ALOX12) were particularly highly expressed in the treatment with combined OR21 and IM. In K562 cells, the gene expression level of LXN was 4.5 times with single drug of OR21, 1.3 times with single drug of IM, and 158.3 times with combination of OR21 and IM in respect of the control, respectively. In KBM5 cells, the gene expression level of LXN was 3.4 times with single drug of OR21, 1.9 times with single drug of IM, and 7.9 times with combination of OR21 and IM in respect of the control, respectively.

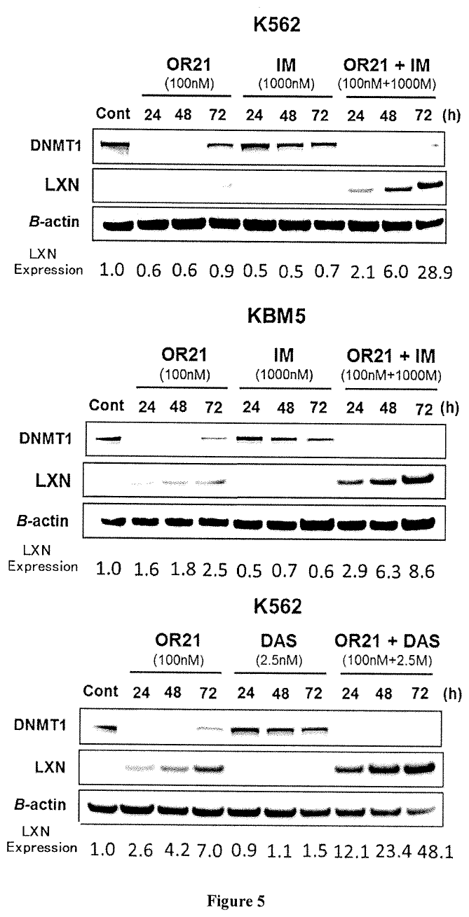

0, 24, and 48 hours after the cell seeding, 100 nM OR21, a DNA methyltransferase inhibitor, was added. Then, 1000 nM imatinib (IM,) or 2.5 nM dasatinib (DAC), a tyrosine kinase inhibitor, was added and further cultured for 1, 2, and 3 days. After the culture, the cells were collected, dissolved, and determined for protein expression level of LXN by Western blotting. In parallel, the cells not added with imatinib after being added with 100 nM OR21 and the cells not added with OR21 but added only with 1000 nM imatinib or 2.5 nM dasatinib were also cultured and determined similarly for the protein expression level of LXN by Western blotting. The results are shown in FIG. 5. The expression level of LXN shown in FIG. 5 represents each expression level of the cases when the LXN expression under each condition was corrected with β actin and the expression level in the control was taken as 1.0. In the cells added with 1000 nM imatinib or 2.5 nM dasatinib, a tyrosine kinase inhibitor, almost no effect on the protein expression level of LXN was observed. However, upon the addition of 100 nM OR21, a DNA methyltransferase inhibitor, an increase in the protein expression level of LXN over time was observed. Furthermore, upon the addition of 1000 nM imatinib or 2.5 nM dasatinib followed by the addition of OR21, a significant increase in protein expression level of LXN was observed compared to the case where only OR21 was added.

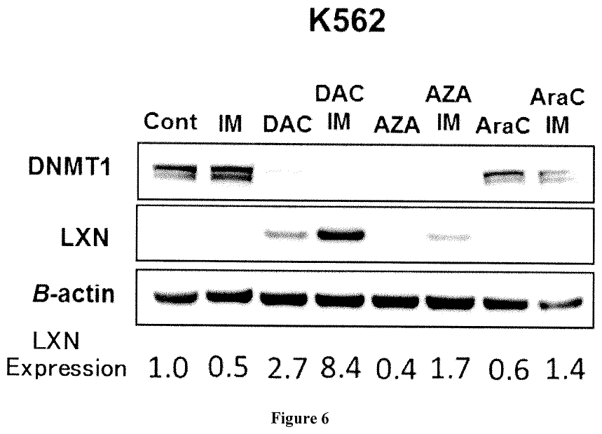

In order to investigate whether increased LXN expression by treatment with the single drug of OR21 or combined OR21 and a TKI is due to DNA demethylation, the protein expression of LXN in cases of the treatment with a single drug of azacytidine (AZA) or decitabine (DAC), another DNA demethylation inhibitor, and a concomitant drug of these DNA demethylation inhibitors and a TKI was examined. In parallel, the protein expression of LXN in cases of the treatment with a single drug of cytarabine (AraC), which has a similar structure to these compounds but does not exhibit DNA demethylation activity, or a concomitant drug of cytarabine and a TKI was examined. 0, 24, and 48 hours after the cell seeding, azacytidine (100 nM), decitabine (100 nM), or cytarabine (100 nM) was added to K562 cell culture, cultured for 2 days. Then imatinib (1000 nM) was added and further cultured for 2 days. In parallel, the cells not added with imatinib after being added with azacytidine (100 nM), decitabine (100 nM), or cytarabine (100 nM), or the cells not added with azacytidine, decitabine, or cytarabine but added only with imatinib (1000 nM) were cultured. After the culture, the cells were collected, dissolved, and determined for protein expression level of LXN by Western blotting. The results are shown in FIG. 6. The expression level of LXN shown in FIG. 6 represents each expression level of the cases when the LXN expression under each condition was corrected with β actin and the expression level in the control was taken as 1.0. Under respective treatment conditions, the DNA demethylating agents azacytidine or decitabine reduced the expression of DNMT1, a DNA methyltransferase, whereas treatment with decitabine increased the protein expression of LXN. In addition, by using azacytidine or decitabine in combination with imatinib, a significant increase in protein expression of LXN was observed compared to the cases of treatment with each of their single drugs. On the other hand, cytarabine did not exhibit inhibitory effect on the expression of DNMT1 and no increase in the expression of LXN was observed in the treatment with the single drug of cytarabine or its concomitant drug with imatinib. Consequently, it was suggested that DNA demethylation activity is important for the increase in the expression of LXN by the combination with a tyrosine kinase inhibitor.

### Example 4

### Gene Expression Levels of LXN in Patients with CML and Healthy Subjects

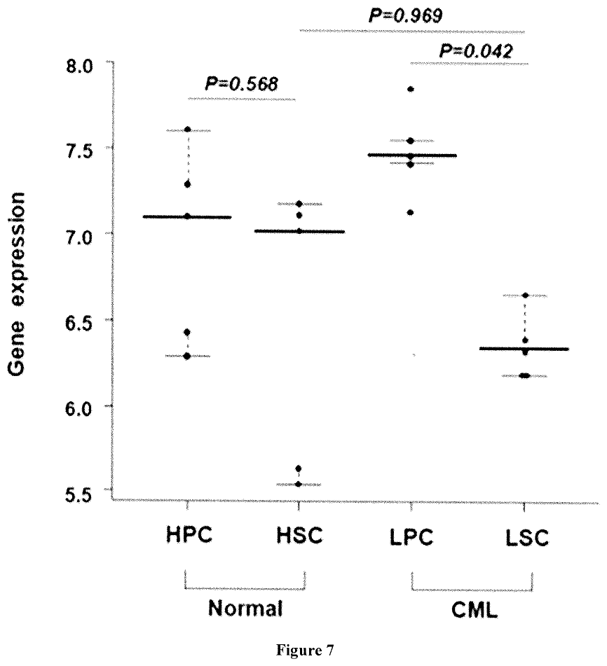

CD34+ lin-cells were concentrated from mononuclear cells isolated from the bone marrow of healthy subjects (Normal: 5 cases), and then separated into hematopoietic progenitor cells (CD34+CD38+lin-cells; HPCs) and hematopoietic stem cells (CD34+CD38-lin-cells; HSCs), respectively. For patients with CML (CML: 5 cases), hematopoietic progenitor cells (CD34+CD38+lin-cells; LPCs) and hematopoietic stem cells (CD34+CD38-lin-cells; LSCs) were separated, respectively with the same method. Total RNA was extracted from these cells (HPCs, HSCs, LPCs, LSCs), respectively and a comprehensive analysis of gene expression levels in these cells was carried out with Affymetrix Human Gene 1.0 ST Array [transcript (gene) version]. The results have been reported by Vazquez S A, Gonzalez A C, Miranda A H et al., entitled “Global gene expression profiles of hematopoietic c stem and progenitor cells from patients with chronic myeloid leukemia: the effect of in vitro culture with or without imatinib.” in Cancer Med. 2017 Dec; 6(12): 2942-56). The present inventors analyzed the results reported by Vazquez et al. and calculated the mRNA expression level of LXN in the above cells. The results are shown in FIG. 7, wherein, in the graph, the centerline indicates values of median, the upper and lower bars indicate 10-90 percentile, and the vertical axis indicates the gene expression level. The p-values calculated with Study's t-tset are indicated. As shown in FIG. 7, in healthy subjects, no difference in LXN expression between hematopoietic progenitor cells and stem cells was observed. However, in patients with CML, hematopoietic stem cells had significantly lower values of LXN expression than hematopoietic progenitor cells, suggesting that LXN expression is low in hematopoietic stem cells from patients with CML.

### Example 5

### Effects of DNA Methyltransferase Inhibitors and Tyrosine Kinase Inhibitors on the Stem Cells of CML

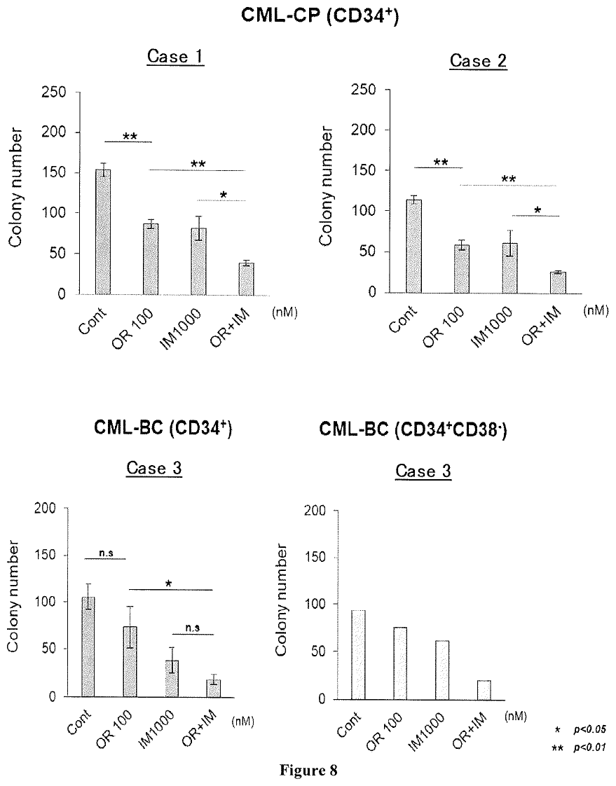

In order to assess the effect on the stem cells of CML by the combination, the ability to form colonies when each drug was added to the cells collected from patients with CML was assessed. CD34+ cells were separated from the myeloid cells of patients with CML (2 cases in chronic phase: CML-CP, or 1 case in blast phase: CML-BC). In addition, for the case of CML-BC (case 3), CD34+CD38-cells were separated from the myeloid cells. The separated cells were placed in an IMDM medium (+20% FBS) and the cell number of the cell suspension was counted. The cell suspension was adjusted to have the cell number of 3,000 with the cell suspension and MethoCult, transferred to a culture dish, and then cultured for 14 days under conditions of 37° C. and 5% CO₂. As the MethoCult used for the addition, one added with each of the drugs to the final concentration of 100 nM OR21 or 1,000 nM imatinib (OR 100 or IM 1000), one added with both (OR+IM), and one not added with either of both (Cont) were used. The colony number after culture for 14 days is shown in FIG. 8, wherein, the bars show standard deviations. Statistical analysis was carried out with Study's t-tset, wherein, * indicates p<0.05, ** indicates p<0.01, and n.s indicates no significant difference. As shown in FIG. 8, the colony number was reduced in the treatment with combined OR21 and imatinib, suggesting that the ability of the stem cells of CML to form colonies is reduced by combination of OR21 and imatinib.

1. An inhibitor against stem cells of chronic myeloid leukemia comprising a compound represented by formula (I) or a salt thereof: 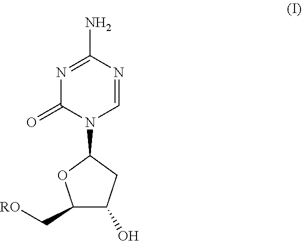
wherein R is a silyl group represented by formula (II): 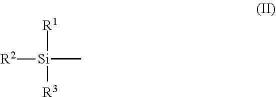
wherein each of R¹, R², and R³ is an alkyl group which may have a substituent.

2. The inhibitor according to claim 1, wherein the alkyl group is a methyl, ethyl, or propyl group.

3. The inhibitor according to claim 2, wherein the alkyl group is an ethyl group.

4. The inhibitor according to claim 1, wherein the compound has the formula:

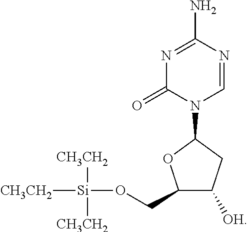

5. A pharmaceutical composition comprising an inhibitor of claim 1 and a pharmaceutically acceptable carrier

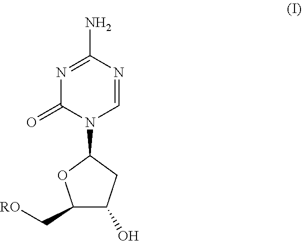

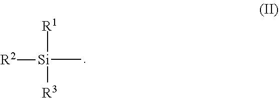

6. (canceled).

7. (canceled).

8. (canceled).

9. The pharmaceutical composition according to claim 5, characterized by being combined with a tyrosine kinase inhibitor.

10. The pharmaceutical composition according to claim 9, wherein the tyrosine kinase inhibitor is one or more selected from the group consisting of imatinib, gefitinib, erlotinib, sorafenib, dasatinib, sunitinib, lapatinib, nilotinib, pazoponib, crizotinib, ruxolitinib, vandertinib, vemurafenib, axitinib, bosutinib, canonzantinib, ponatinib, regorafenib, tofacitinib, afatinib, dabrafenib, ibrutinib, trametinib, ceritinib, nintedanib, lenvatinib, palbocitinib, carbozantinib, aclabrutinib, brigatinib, neratinib, dacomitinib, gilteritinib, larotrectinib, lorlatinib, and osimertinib.

11. The pharmaceutical composition according to claim 9, wherein the tyrosine kinase inhibitor is one or more selected from the group consisting of imatinib, nilotinib, dasatinib, bosutinib, and ponatinib.

12. The pharmaceutical composition according to claim 9, wherein the compound has the formula: 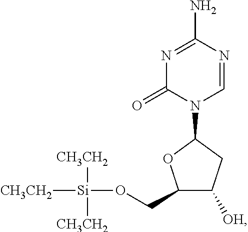
and wherein the tyrosine kinase inhibitor is one or more selected from the group consisting of imatinib, nilotinib, dasatinib, bosutinib, and ponatinib.

13. (canceled).

14. The pharmaceutical composition according to claim 9, wherein the composition can be administered by oral administration, parenteral administration, or a combination thereof.

15. The pharmaceutical composition according to claim 9, wherein the inhibitor represented by formula (I) or a salt thereof is administered orally and the tyrosine kinase inhibitor is administered orally or parenterally.

16. The pharmaceutical composition according to claim 5 for preventing the recurrence of chronic myeloid leukemia that occurs after treatment with a tyrosine kinase inhibitor is discontinued followed by amelioration due to the treatment of chronic myeloid leukemia with the drug.

17. A therapeutic method for chronic myeloid leukemia, comprising a step of administering to a patient in need of the treatment of chronic myeloid leukemia, a pharmaceutically effective amount of an inhibitor of claim 1 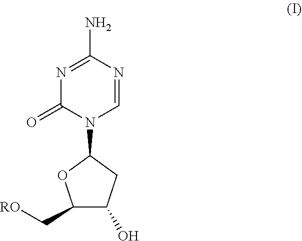 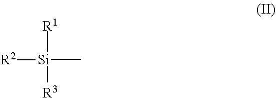
wherein the method prevents the recurrence of chronic myeloid leukemia based on the effect of inhibiting stem cells of chronic myeloid leukemia.

18. The therapeutic method according to claim 17, wherein the alkyl group is a methyl, ethyl, or propyl group.

19. The therapeutic method according to claim 18, wherein the alkyl group is an ethyl group.

20. The therapeutic method according to claim 17, wherein the compound has the formula:

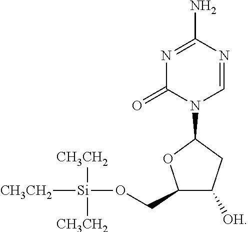

21. The therapeutic method according to claim 17, in combination with a tyrosine kinase inhibitor.

22. The therapeutic method according to claim 21, wherein the tyrosine kinase inhibitor is one or more selected from the group consisting of imatinib, gefitinib, erlotinib, sorafenib, dasatinib, sunitinib, lapatinib, nilotinib, pazoponib, crizotinib, ruxolitinib, vandertinib, vemurafenib, axitinib, bosutinib, canonzantinib, ponatinib, regorafenib, tofacitinib, afatinib, dabrafenib, ibrutinib, trametinib, ceritinib, nintedanib, lenvatinib, palbocitinib, carbozantinib, aclabrutinib, brigatinib, neratinib, dacomitinib, gilteritinib, larotrectinib, lorlatinib, and osimertinib.

23. The therapeutic method according to claim 21, wherein the tyrosine kinase inhibitor is one or more selected from the group consisting of imatinib, nilotinib, dasatinib, bosutinib, and ponatinib.

24. The therapeutic method according to claim 21, wherein the compound has the formula: 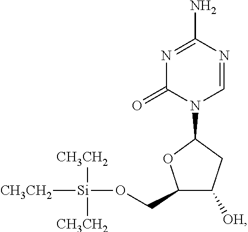
and wherein the tyrosine kinase inhibitor is one or more selected from the group consisting of imatinib, nilotinib, dasatinib, bosutinib, and ponatinib.

25. The therapeutic method according to claim 21, wherein the compound represented by formula (I) or a salt thereof is administered after administration of the tyrosine kinase inhibitor in treating a patient with chronic myeloid leukemia.

26. The therapeutic method according to claim 21, wherein the administration comprises oral administration, parenteral administration, or a combination thereof.

27. A therapeutic method according to claim 21, wherein the compound represented by formula (I) or a salt thereof is administered orally and the tyrosine kinase inhibitor is administered orally or parenterally.

28. A therapeutic method according to claim 17 for preventing the recurrence of chronic myeloid leukemia that occurs after treatment with a tyrosine kinase inhibitor is discontinued followed by amelioration due to the treatment of chronic myeloid leukemia.

29. A method for assessing the efficacy of treatment with a drug in a patient with chronic myeloid leukemia, comprising a step of determining the expression level of latexin in a sample obtained from the patient during or after the treatment with the drug, a step of comparing these levels of expression, and a step of assessing the efficacy of the treatment with the drug, wherein
(1) when the expression level of latexin during or after the treatment with the drug has increased compared to that before the treatment, it is assessed that the treatment with the drug in the patient is effective and the treatment with the drug can be discontinued or terminated without recurrence of the disease,
(2) when the expression level of latexin during or after the treatment with the drug does not increase compared to that before the treatment, it is assessed that the disease will relapse after the treatment with the drug is discontinued or terminated, or
(3) when the assessment of (2) is made, it is assessed that it is effective in preventing recurrence of the disease to continue the treatment with the drug in combination with a drug having an inhibitory effect on the stem cells of chronic myeloid leukemia, or continue the treatment with a drug having an inhibitory effect on the stem cells of chronic myeloid leukemia.

30. The method according to claim 29, wherein the sample obtained from the patient is bone marrow or peripheral blood.

31. The method according to claim 29, wherein the increase in the expression level of latexin during or after treatment is 1.5 times or more, preferably 2.0 times or more compared to that before the treatment.

32. The method according to claim 29, wherein the expression level of latexin is the mRNA expression level of latexin.

33. The method according to claim 29 wherein the mRNA expression level is determined using a method selected from the group consisting of RT-PCR, gene expression profiling, and microarray analysis.

34. The method according to claim 29, wherein the expression level of latexin is the protein expression level of latexin.

35. The method according to claim 29, wherein the protein expression level is determined using a method selected from the group consisting of immunohistochemistry, immunofluorescence, mass spectrometry, flow cytometry, and Western blotting.

36. The method according to claim 29, wherein the drug used in the treatment of chronic myeloid leukemia is a tyrosine kinase inhibitor or a compound having the formula:

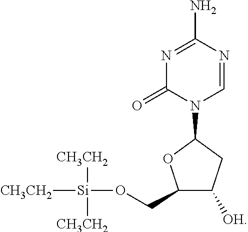

37. The method according to claim 29 wherein the drug having an inhibitory effect on the stem cells of chronic myeloid leukemia is a compound having the formula: [chemical structure]
